(19) United States
(12) Patent Application Publication
(10) Pub. No.: US 2025/0001142 A1
(43) Pub. Date: Jan. 2, 2025

(54) BALLOON CATHETER FOR BULB INDUCTION

(71) Applicant: NORDLANDSSYKEHUSET HF, Bodø (NO)

(72) Inventors: Stine Andreasen, Bodø (NO); Hege Hansen, Bodø (NO)

(21) Appl. No.: 18/710,478

(22) PCT Filed: Jan. 23, 2023
(86) PCT No.: PCT/NO2023/050016

§ 371 Date: May 15, 2024

(30) Foreign Application Priority Data
Jan. 26, 2022 (NO) 20220130

(51) Int. Cl. A61M 25/10

(52) U.S. Cl. CPC A61M 25/1018; A61M 25/1002

(57) ABSTRACT

The present invention relates to a balloon catheter used in connection with childbirth. The balloon catheter includes a liquid supply tube 7 and a mandrin guide tube 4 extending parallel to the liquid supply tube 7. A liquid supply connecting portion 8 is connected to a first end of the liquid supply tube 7, and the first end of the liquid supply tube 7 with the liquid supply connecting portion 8 is adapted to be connected to a liquid supply element. A balloon 2 with a continuous outer surface apart from a tube connecting portion 14 is connected to a second end of the liquid supply tube 7. The mandrin guide tube 4 and the liquid supply tube 7 are flexible and foldable allowing the first end of the mandrin guide tube 4 and the liquid supply tube 7 to be folded and inserted into a vagina of a user.

The present invention relates to a bulb catheter for bulb induction to induce childbirth. The bulb catheter may be a Prefilled Induction Catheter, (PIC).

Some women find the insertion of a bulb/balloon catheter (hereinafter balloon catheter) unpleasant and painful. Furthermore, does the use of traditional balloon catheters involve allowing a portion of the balloon catheter to be on the outside of the vagina, and this portion is typically taped to the thigh. This practice is by some considered as disgraceful and unpleasant. Traditional balloon catheters also appear as typical medical equipment that is considered unappealing by some.

The invention seeks to simplify and improve equipment used for bulb induction during childbirth and in that way contribute to improve the experience for the mother when giving birth.

It is an aim to reduce the time required to insert the balloon catheter, to reduce the discomfort during implementation and use, to provide a solution without any parts that extend out of the vagina when implemented, that gives an appealing visual impression, that is safe and hygienic, that is easy to operate for the doctor or midwife, that is simple to remove and that reduces the risk of spillage of the saltwater solution.

The present invention is provided to alleviate some of the problems with the prior art and discloses a balloon catheter for balloon induction used in connection with childbirth.

The balloon catheter includes a liquid supply tube and a mandrin guide tube extending parallel to the liquid supply tube. A liquid supply connecting portion is attached to a first end of the liquid supply tube. The first end of the liquid supply tube with the liquid supply connecting portion is adapted to be connected to a liquid supply element. A balloon with a continuous outer surface apart from a tube connecting portion is in liquid connection with a second end of the liquid supply tube.

The mandrin guide tube and the liquid supply tube may be flexible and foldable.

The mandrin guide tube and the liquid supply tube may be adapted to allow the first end of the mandrin guide tube and the liquid supply tube to be folded and inserted into a vagina of a user.

The mandrin guide tube may be a separate tube with a larger inner diameter than the liquid supply tube, and the liquid supply tube may extend through the mandrin guide tube.

The liquid supply connecting portion may be a check valve.

The liquid supply connecting portion may include a flexible element with a slit that is normally sealed to prevent fluid from escaping through the liquid supply connecting portion while allowing a tube to be inserted through the slit to inject or withdraw fluid through the flexible element.

The the liquid supply connecting portion may include an outer sleeve inserted into the first end of the liquid supply tube.

The the liquid supply connecting portion may include a penetrable flexible element projecting out of the outer sleeve.

The liquid supply element may be a squeezable fluid container with a tube connection.

The liquid supply element may include a hand pump and a tube connection.

The tube connection may include a fluid valve that is adapted to open upon connection with the liquid supply tube.

The balloon catheter may further include a mandrin with a gripping portion and a rod-shaped portion inserted into the mandrin guide tube.

The mandrin may include marking notches to indicate the position of the balloon catheter.

The balloon catheter may further include an inner ball inside the balloon, connected to the second end of the liquid supply tube.

The inner ball may be connected to the second end of the liquid supply tube through a guiding tube attached to the the inner ball and extending into the liquid supply tube.

The balloon may include a balloon fold along the guiding tube when the balloon is in the deflated state to maintain the balloon in a compact configuration for insertion before inflation while ensuring enough material for the balloon expand.

The balloon catheter may further include an inner fluid channel extending through the guiding tube and the inner ball.

### BRIEF DESCRIPTION OF THE ENCLOSED DRAWINGS

FIG. 1 is a side view of a balloon catheter of the invention;

FIG. 2 is a top view of the balloon catheter as shown in FIG. 1;

FIG. 3 is an end view of the balloon catheter as shown in FIGS. 1 and 2;

FIG. 4 is a perspective view of a squeezable fluid container;

FIG. 5 is a perspective view of the balloon catheter of the invention;

FIG. 6 is a perspective view of a mandrin;

FIG. 7 shows a cross-section of an inner part of a balloon catheter of the invention in an uninflated state;

FIG. 8 shows a cross-section of an inner part of a balloon catheter of the invention in an inflated state;

FIG. 9, shows a cross-section of the squeezable fluid container with the tube connection including the cylindrical friction portion;

FIG. 10, is a perspective view of the squeezable fluid container with the tube connection including the cylindrical friction portion; and

FIG. 11 shows a cross-section in part of an alternative embodiment of a liquid supply element.

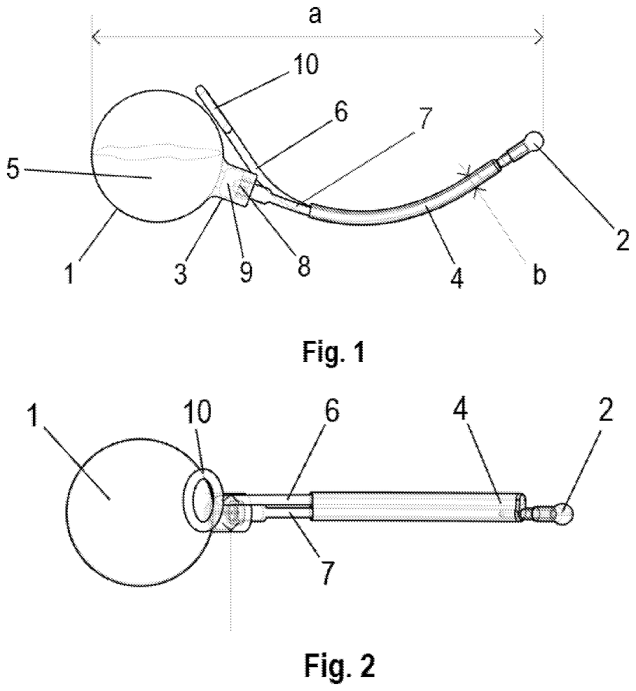

FIG. 1 is a side view of a balloon catheter of the invention. A balloon shaped squeezable fluid container 1 with a saltwater solution 5 includes a tube connection 3 and a fluid valve 9. A liquid supply tube 7 with a liquid supply connecting portion 8 is releasably connected to the squeezable fluid container 1 in the tube connection 3 at a first end and to an expandable bulb or balloon 2 at the other end. The tube connection 3 may include a small tube that may be used to penetrate the liquid supply connecting portion 8 and may include a flexible element 13 with a slit that is normally sealed to prevent fluid from escaping through the liquid supply connecting portion 8 while allowing a tubular portion/small tube to be inserted through the slit to inject or withdraw fluid through the flexible element. A large syringe, rectal syringe or a hand pump with a tubular portion added may be used in place of the squeezable fluid container 1. The mandrin guide tube 4 surrounding the liquid supply tube 7 extends along a portion of the length of the liquid supply tube 7 and serves as a guide for a mandrin 6 with a gripping portion 10 at an end facing the squeezable fluid container 1. The mandrin 6 is slidingly installed in the mandrin guide tube 4 and can be pulled out of the mandrin guide tube by pulling the gripping portion 10. The mandrin 6 makes the assembly of the mandrin 6, the liquid supply tube 7 and the mandrin guide tube 4 stiff to facilitate insertion of the balloon 2 through the vagina, through the cervix and to a location adjacent the fetus. The mandrin 6 may then be removed and the liquid supply tube 7 and the mandrin guide tube 4 may be folded and inserted into the vagina of the uses to allow the balloon catheter to be completely concealed inside the body. The mandrin 6 and the liquid supply tube 7 are thus flexible and foldable hoses. The squeezable fluid container 1 typically holds a volume of 80 ml of the saltwater solution 5. The overall length a of the balloon catheter is typically 206 mm and the outer diameter b of the mandrin guide tube 4 is typically 6 mm. The liquid supply connecting portion 8 may include a one-way valve allowing the saltwater solution 5 to flow from the squeezable fluid container 1 and into the balloon 2 to inflate the balloon 2 when the squeezable fluid container 1 is squeezed. The fluid valve 9 seals the squeezable fluid container 1 when the liquid supply tube 7 is pulled out of and released from the tube connection 3 to prevent spillage of any remaining saltwater solution 5 when the squeezable fluid container 1 is removed. The balloon 2 is flexible to allow inflation with the saltwater solution. The balloon 2 is designed to accept 80 ml of saltwater solution 5.

FIG. 2 is a top view of the balloon catheter shown in FIG. 1. The mandrin 6 extends inside the mandrin guide tube 4, parallel to the liquid supply tube 7. The balloon 2 is uninflated and the liquid supply tube 7 is connected to the squeezable fluid container 1. The gripping portion 10 at the end of the mandrin 6 is shaped as a ring, typically dimensioned to allow a finger to extend through the ring to facilitate the pulling of the mandrin 6 out of the mandrin guide tube 4.

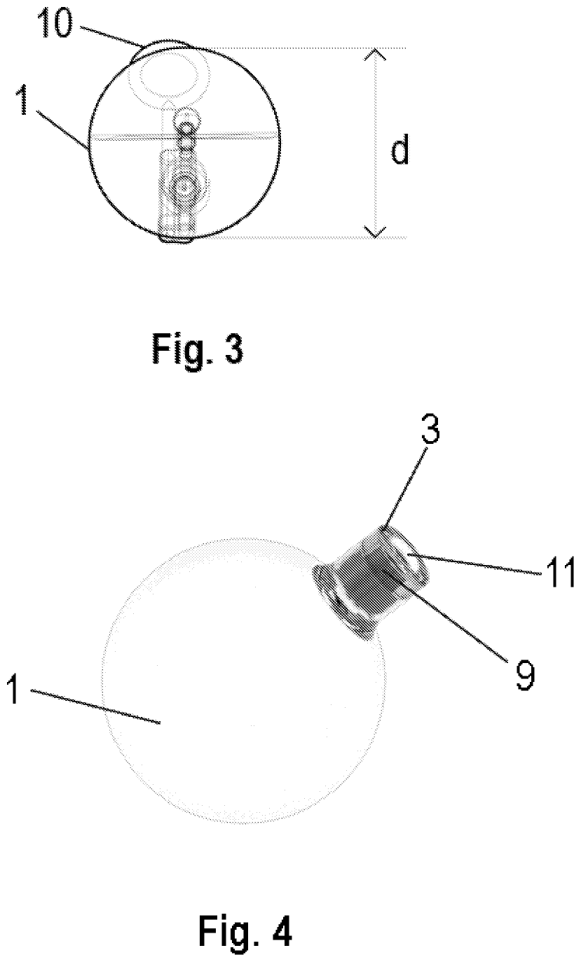

FIG. 3 is an end view of the balloon catheter shown in FIGS. 1 and 2, with the squeezable fluid container 1 partly transparent. The diameter d of the squeezable fluid container 1 is typically 60 mm. The ring-shaped gripping portion 10 is partly extending above the squeezable fluid container 1.

FIG. 4 is a perspective view of the squeezable fluid container 1 with the tube connection 3, the fluid valve 9 and a cylindrical friction portion 11 forming a pressure fit with the liquid supply tube. This tube connection is in place of or in addition to the thin tube. The tube connection 3 could involve other types of connections such as a screw connection or a bayonet connection. It is however a major advantage if the liquid supply tube does not include any bulky connecting elements as the liquid supply tube ideally can be folded and inserted into a vagina to avoid any elements extending out of the vagina. Any elements that could cause discomfort or that runs a risk of penetrating the skin should thus be avoided. The squeezable fluid container 1 is typically a disposable container that is supplied prefilled with a saltwater solution.

The squeezable fluid container 1 is typically made of a soft and elastomeric material to allow the squeezable/compressible fluid container to be squeezed manually to eject the fluid from the container.

The fluid valve 9 may be of a type that opens when the tube connecting portion is inserted into the tube connection 3.

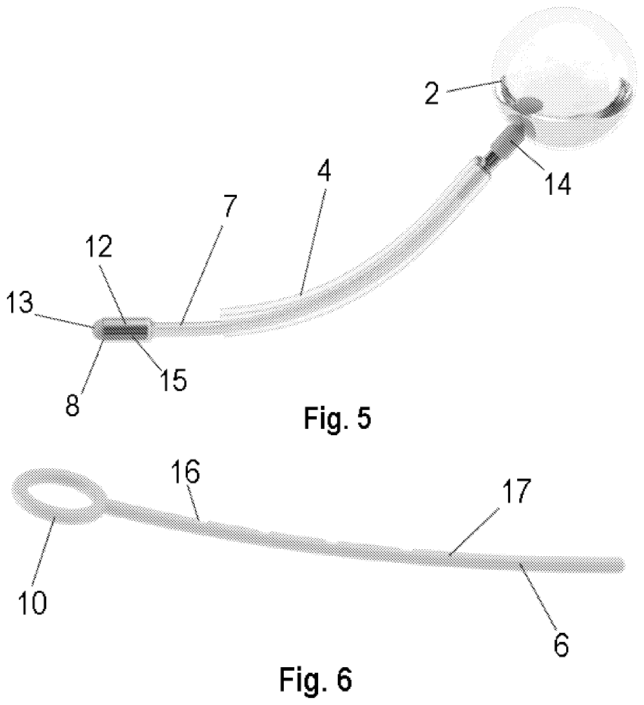

FIG. 5 shows the balloon catheter of the invention without the squeezable fluid container and with the balloon 2 inflated. The balloon is connected to the liquid supply tube 7 at a tube connecting portion 14. The balloon 2 thus represents the outer end portion of the balloon catheter and has a smooth outer surface. The balloon catheter thus has no protruding elements beyond the balloon 2, and the liquid supply tube 7 or the mandrin does not extend through the balloon 2. The mandrin is withdrawn from the mandrin guide tube 4 when the balloon has been inserted and inflated. The liquid supply connecting portion 8 may include an outer sleeve 15 that also serves to produce a stiffened tube connecting portion 12 forming a press fit with the tube connection of the squeezable fluid container. The liquid supply connecting portion 8 may include a check valve/one-way valve in place of a penetrable flexible element 13 and may include an outer sleeve 15. The penetrable flexible element 13 projects from the outer sleeve 15 to enable the penetrable flexible element 13 to be penetrated with a tubular object to deflate the balloon 2 (for instance for cessation of the treatment). The outer sleeve 15 is pressed into the liquid supply tube 7 and the soft material of the liquid supply tube 7 will seal against the cylindrical friction portion of the tube connection of the squeezable fluid container.

The balloon is typically made of an elastomeric material with a considerable elasticity to enable the balloon to expand to the required size.

FIG. 6 is a perspective view of the mandrin 6 with the ring-shaped gripping portion 10 and the rod-shaped portion 17. Indentations or marking notches 16 indicates how far the mandrin 6 is pulled out of the mandrin guide tube 4. The marking notches 16 can also be seen or felt through the mandrin guide tube and can be used to assess how far in the balloon catheter is inserted. The mandrin 6 is typically made of a relatively stiff plastic material.

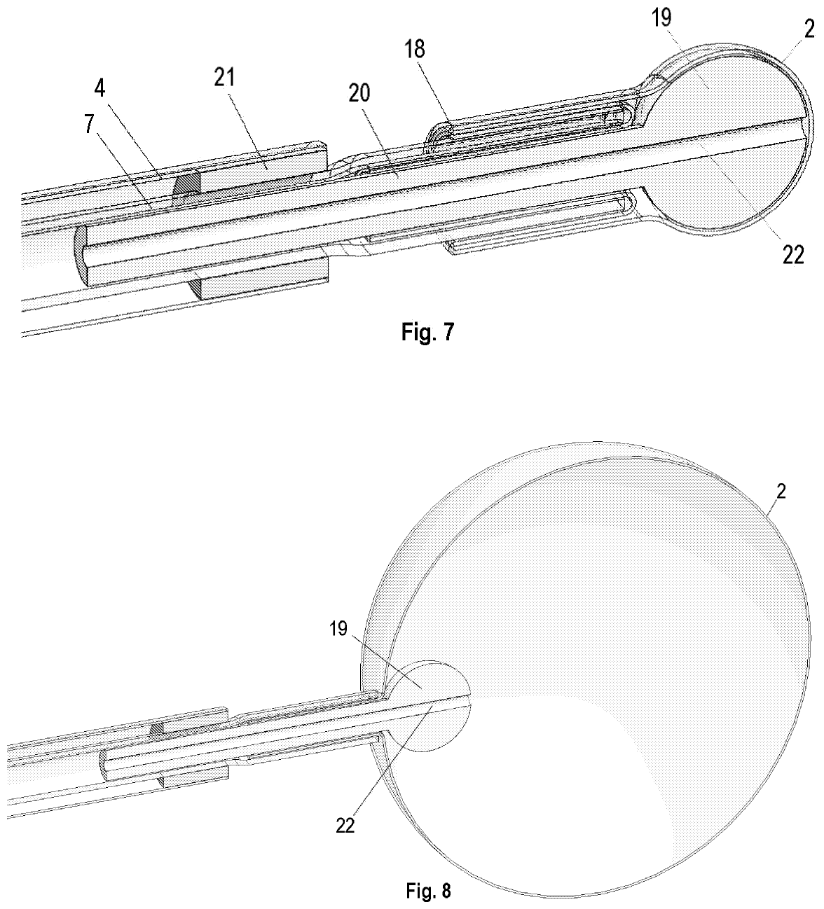

FIG. 7 is a perspective view of a cross section of an inner end of a balloon catheter of the invention. The balloon 2 is uninflated and is located on the outside of an inner ball 19 secured to a guiding tube 20. An inner fluid channel 22 extends through the inner ball 19 and the guiding tube 20. The guiding tube 20 extends into the liquid supply tube 7. The balloon 2 includes a balloon fold 18 to maintain the balloon 2 in a compact configuration for insertion before inflation while ensuring enough material for the balloon. The inner ball 19 provides a smooth outer surface of the balloon 2 before it is inflated to reduce discomfort during insertion while the risk of puncturing the balloon is reduces. The inner ball also forms a guide and a reinforcement, making it easier to enter cervix. The inner ball does not have to be completely round but could also for instance have a slightly elongated shape or tapered shape.

A mandrin end stop 21 at the end of the mandrin guide tube 4 ensures that the mandrin not extends out of the mandrin guide tube 4 and that the mandrin is effective for inserting the balloon catheter.

FIG. 8 corresponds to FIG. 7 but shows the balloon 2 in an inflated state and a major part of the balloon 2 is no longer in contact with the inner ball 19. The balloon 2 is inflated with liquid through the inner fluid channel 22 in the inner ball 19 and the guiding tube 20. The guiding tube 20 ensures that the assembly of the balloon catheter and the mandrin form a stiff assembly at the inner end to facilitate insertion.

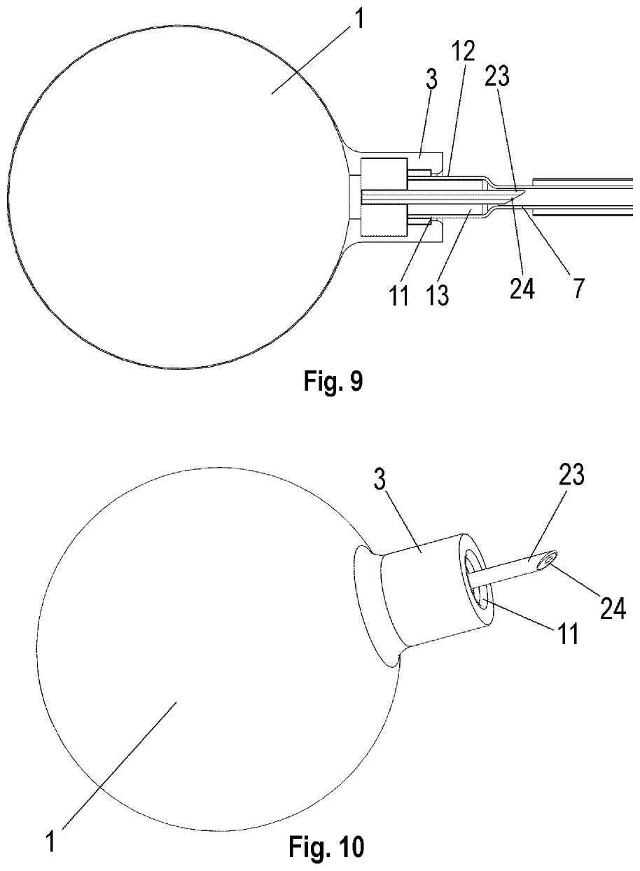

FIG. 9, is a cross section of the squeezable fluid container 1 with the tube connection 3 including the cylindrical friction portion 11. The penetrable flexible element 13 is inserted into the liquid supply tube 7 and forms the tube connecting portion 12 extending into the cylindrical friction portion 11 to hold the squeezable fluid container 1 to the liquid supply tube 7 and to reinforce the transition between the two. A connecting tube 23 with a pointed end 24 extends through the penetrable flexible element 13 to allow the squeezable fluid container 1 to fill or drain the balloon catheter. The penetrable flexible element 13 seals the liquid supply tube 7 when the connecting tube 23 is pulled out of the penetrable flexible element 13. The tube connecting portion 12 extending into the cylindrical friction portion 11 reduces the stresses on the connecting tube 23 when the squeezable fluid container 1 is in use. The penetrable flexible element 13 inserted into the liquid supply tube 7 in combination with the cylindrical friction portion 11 reduces the risk of kinking the liquid supply tube 7.

FIG. 10, is a perspective view of the squeezable fluid container 1 with the tube connection 3 including the cylindrical friction portion 11, and the connecting tube 23 with the pointed end 24. The connecting tube 23 is typically a blunt plastic tube. The pointed end 24 facilitates connection of the squeezable fluid container 1 to the balloon catheter. The plastic connecting tube 24 is considered to be safe and prevents inadvertently stabbing the patient, the personnel or puncturing the balloon. It is thus an advantage not using a hypodermic needle.

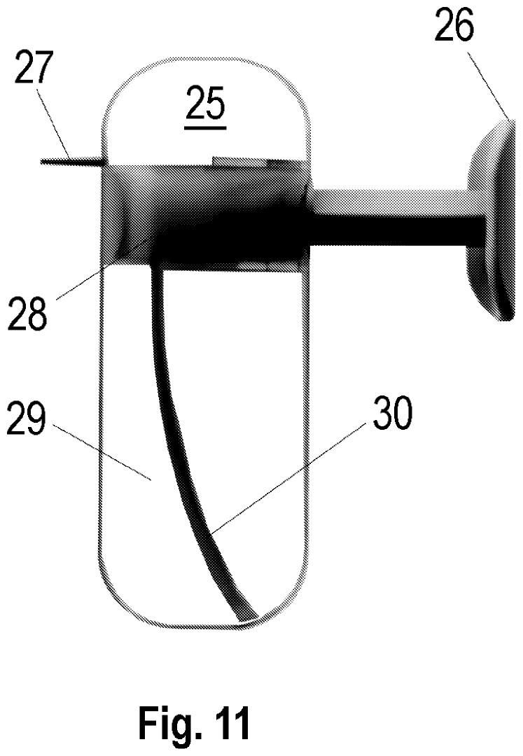

FIG. 11 is a cross-sectional view of an alternative embodiment of a liquid supply element 25 with a liquid tank 29 and a hand pump 28 with a spring-loaded squeeze handle 26 actuating the pump 28. A pickup tube 30 extends from the pump and to the bottom of the liquid tank 29. A liquid supply tube connection 27 is adapted to be connected to the liquid supply tube. A flow path is thus formed from the liquid tank 29, through the pickup tube 30, through the pump 28, through the liquid supply tube connection 27, and into the liquid supply tube. The hand pump 28 typically includes a hand operated piston or diaphragm and one or several check valves.

The liquid supply tube connection 27 is shown as a tapered spout or nozzle corresponding to the connecting tube 23 shown in FIGS. 9, 10. Other connections may however be utilized.

The design of FIG. 11 allows a higher liquid pressure to be provided by the liquid supply element 25 compared to the squeezable fluid container shown in FIGS. 1-4.

The hand pump 28 is operated by the spring-loaded squeeze handle 26 and the liquid supply element 25 may include two modes of operation to enable the liquid supply element 25 to both fill and empty the balloon. Again, the liquid inside the liquid supply element 25 is typically salt water, and the element may be a single use element or a refillable element. A volume of the liquid tank 29 may correspond to a maximum volume of the balloon to prevent any overfilling of the balloon and is typically 80 ml. As this embodiment also enables a higher pressure in the system may a limitation of the available volume ensure that the size of the balloon not exceeds a certain value and that the balloon is prevented from bursting. The higher pressure of this system also enables inflation of a more robust balloon compared to the lower pressure system with the squeezable fluid container of FIGS. 1-4.

Alternative liquid supply elements may clearly be utilized, and a hand pump with a lever instead of the spring-loaded squeeze handle is well known within the art of hand pumps. Other liquid supply elements may include an electric powered liquid pump or a source of compressed air/gas applying pressure to the liquid tank to expel the liquid.

Using air to inflate the balloon is less favourable as a leak would be uncomfortable and an accidental rupture of the balloon more unfortunate and even harmful. The added mass of the water is also favourable for the induction.

The balloon catheter for bulb induction of the invention may be an inexpensive and disposable/recyclable balloon catheter to ensure ease of use and good hygiene. The present invention does not exclude a further liquid tube extending in parallel to the liquid supply tube to facilitate draining and deflation of the balloon.

Clearly, the various features of the different embodiments may be combined, and for instance the tube connection shown in relation to the squeezable fluid container may clearly be utilized in the embodiment with a hand pump.

| | |
|---|---|
| 1 | Squeezable fluid container |
| 2 | balloon |
| 3 | Tube connection |
| 4 | Mandrin guide tube |
| 5 | Saltwater |
| 6 | Mandrin |
| 7 | Liquid supply tube |
| 8 | Tube valve/liquid supply |
| | connecting portion |
| 9 | Fluid valve |
| 10 | Gripping portion |
| 11 | Cylindrical friction portion |
| 12 | Tube connecting portion |
| 13 | Penetrable flexible element |
| 14 | Tube connecting portion |
| 15 | Outer sleeve |
| 16 | Marking notches |
| 17 | Rod shaped portion |
| 18 | Fold of balloon |
| 19 | Inner ball |
| 20 | Guiding tube |
| 21 | Mandrin end stop |
| 22 | Inner fluid channel |
| 23 | Connecting tube |
| 24 | Pointed end |
| 25 | Liquid supply element |
| 26 | Spring-loaded squeeze handle |
| 27 | Liquid supply tube connection |
| 28 | Hand pump |
| 29 | Liquid tank |
| 30 | Pickup tube |

1. A balloon catheter for balloon induction in connection with childbirth comprising:
a liquid supply tube;
a mandrin guide tube extending parallel to the liquid supply tube;
a liquid supply connecting portion attached to a first end of the liquid supply tube, and wherein the first end of the liquid supply tube with the liquid supply connecting portion is configured to be connected to a liquid supply element; and
a balloon configured to accommodate a deflated mode and an expanded liquid filled mode, and with a continuous outer surface apart from a tube connecting portion in liquid connection with a second end of the liquid supply tube.

2. The balloon catheter of claim 1, wherein the mandrin guide tube and the liquid supply tube are flexible and foldable, whereby the mandrin guide tube and the liquid supply tube are configured to allow the first end of the mandrin guide tube and the liquid supply tube to be folded and inserted into a vagina of a user.

3. The balloon catheter of claim 1, wherein the mandrin guide tube is a separate tube with a larger inner diameter than the liquid supply tube; and wherein the liquid supply tube extends through the mandrin guide tube.

4. The balloon catheter of claim 1, wherein the liquid supply connecting portion is a check valve.

5. The balloon catheter of claim 1, wherein the liquid supply connecting portion includes a flexible element with a slit that is normally sealed to prevent fluid from escaping through the liquid supply connecting portion while allowing a tube to be inserted through the slit to inject or withdraw fluid through the flexible element.

6. The balloon catheter of claim 1, wherein the liquid supply connecting portion includes an outer sleeve inserted into the first end of the liquid supply tube.

7. The balloon catheter of claim 4, wherein the liquid supply connecting portion includes a penetrable flexible element projecting out of the outer sleeve.

8. The balloon catheter of claim 1, wherein the liquid supply element is a squeezable fluid container with a tube connection.

9. The balloon catheter of claim 1, wherein the liquid supply element includes a hand pump and a tube connection.

10. The balloon catheter of claim 8, wherein the tube connection includes a fluid valve that is configured to open upon connection with the liquid supply tube.

11. The balloon catheter of claim 1, further including a mandrin with a gripping portion and a rod-shaped portion inserted into the mandrin guide tube.

12. The balloon catheter of claim 11, wherein the mandrin includes marking notches to indicate the position of the balloon catheter.

13. The balloon catheter of claim 1, further including an inner ball inside the balloon, connected to the second end of the liquid supply tube.

14. The balloon catheter of claim 13, wherein the inner ball is connected to the second end of the liquid supply tube through a guiding tube attached to the the inner ball and extending into the liquid supply tube.

15. The balloon catheter of claim 13, wherein the balloon includes a balloon fold along the guiding tube when the balloon is in the deflated state to maintain the balloon in a compact configuration for insertion before inflation while ensuring enough material for the balloon to expand.

16. The balloon catheter of claim 13, further including an inner fluid channel extending through the guiding tube and the inner ball.
